(19) United States
(12) Patent Application Publication
(10) Pub. No.: US 2004/0005617 A1
(43) Pub. Date: Jan. 8, 2004

(54) SERPINB13 single nucleotide polymorphisms and treatment of cancer

(76) Inventors: Wun-Shaing Wayne Chang, Taipei (TW); Cheng-Wen Wu, Taipei (TW)

Correspondence Address: FISH & RICHARDSON PC, 225 FRANKLIN ST, BOSTON, MA, 02110, US

(21) Appl. No.: 10/444,748

(22) Filed: May 23, 2003

Related U.S. Application Data
(60) Provisional application No. 60/382,924, filed on May 24, 2002.

(51) Int. Cl. C12Q 1/68

(52) U.S. Cl. 435/6

(57) ABSTRACT

A method of determining whether a subject is suffering from or at risk for developing cancer. The method includes preparing a nucleic acid sample from a subject, and identifying a single nucleotide polymorphism in the SERPINB13 gene. The presence of a single nucleotide polymorphism indicates that the subject is suffering from or at risk for developing cancer. Also disclosed is a method of treating cancer by administering to a subject in need thereof an effective amount of a nucleic acid encoding the Hurpin protein or an effective amount of the Hurpin protein.

### RELATED APPLICATION INFORMATION

This application claims priority to U.S. provisional application serial No. 60/382,924, filed May 24, 2002.

### BACKGROUND

Single nucleotide polymorphisms (SNPs) have been known to be associated with cancer. However, very few cancer-associated SNPs have been identified.

SERPINB13 (SERine Protease INhibitor, clade B, member 13), a newly identified gene encoding a protease inhibitor Hurpin or Headpin, contains 8 exons separated by 7 introns. More specifically, Exon 1 contains the 5′-UTR, Exon 2 contains the translation start site, and Exon 8 is the pivotal region that determines the specificity of protease inhibition. Two SNPs have been identified within Exon 8: (1) an adenine to guanine change at nucleotide 877 of the SERPINB13 coding region that changes amino acid 293 from serine to glycine; and (2) a silent thymine to cytosine change at nucleotide 1047 of the SERPINB13 coding region that does not alter amino acid 349 (threonine).

### SUMMARY

The present invention relates to methods of diagnosing and treating cancer associated with SERPINB13 or its SNPs.

In one aspect, this invention features a method of determining whether a subject is suffering from or at risk for developing cancer. The method includes preparing a nucleic acid sample from a subject and identifying an SNP in the SERPINB13 gene. The presence of an SNP indicates that the subject is suffering from or at risk for developing cancer.

Examples of SNPs include a nucleotide change from adenine to guanine, thymine, or cytosine at position 877 of SEQ ID NO:1 and a nucleotide change from thymine to cytosine, adenine, or guanine at position 1047 of SEQ ID NO:1.

In the case of brain cancer, for example, a guanine can be present at nucleotide 877 of SEQ ID NO:1 or a thymine can be present at nucleotide 1047 of SEQ ID NO:1, and the subject (e.g., a male) can be at least twenty-eight years old.

In another aspect, this invention features a method of treating cancer (e.g., brain or ovarian cancer, or invasive or metastatic cancer). The method can be a gene therapy treatment, including administering to a subject (e.g., a cancer patient) in need thereof an effective amount of a nucleic acid containing SEQ ID NO:1, thereby providing a functional Hurpin protein. Another method includes administering to a subject in need thereof an effective amount of a polypeptide encoded by SEQ ID NO:1. A polypeptide functionally equivalent to a naturally occurring Hurpin protein (e.g., a fragment of a naturally occurring Hurpin protein) and a nucleic acid encoding such a polypeptide are within the scope of this invention.

The present invention provides methods that can be used for cancer marker carrier identification, newborn screening, prenatal diagnosis, and cancer treatment. The details of one or more embodiments of the invention are set forth in the accompanying description below. Other advantages, features, and objects of the invention will be apparent from the detailed description, and from the claims.

### DETAILED DESCRIPTION

SERPINB13 gene encodes a protease inhibitor. The coding region of the human SERPINB13 gene is shown below:

| | | |
|---|---|---|
| 1 | ATGGATTCAC TTGGCGCCGT CAGCACTCGA CTTGGGTTTG ATCTTTTCAA | (SEQ ID NO:1) |
| 51 | AGAGCTGAAG AAAACAAATG ATGGCAACAT CTTCTTTTCC CCTGTGGGCA | |
| 101 | TCTTGACTGC AATTGGCATG GTCCTCCTGG GGACCCGAGG AGCCACCGCT | |
| 151 | TCCCAGTTGG AGGAGGTGTT TCACTCTGAA AAAGAGACGA AGAGCTCAAG | |
| 201 | AATAAAGGCT GAAGAAAAAG AGGTGATTGA GAACACAGAA GCAGTACATC | |
| 251 | AACAATTCCA AAAGTTTTTG ACTGAAATAA GCAAACTCAC TAATGATTAT | |
| 301 | GAACTGAACA TAACCAACAG GCTGTTTGGA GAAAAAACAT ACCTCTTCCT | |
| 351 | TCAAAAATAC TTAGATTATG TTGAAAAATA TTATCATGCA TCTCTGGAAC | |
| 401 | CTGTTGATTT TGTAAATGCA GCCGATGAAA GTCGAAAGAA GATTAATTCC | |
| 451 | TGGGTTGAAA GCAAAACAAA TGAAAAAATC AAGGACTTGT TCCCAGATGG | |
| 501 | CTCTATTAGT AGCTCTACCA AGCTGGTGCT GCTGAACATG GTTTATTTTA | |
| 551 | AAGGGCAATG GGACAGGGAG TTTAAGAAAG AAAATACTAA GGAAGAGAAA | |
| 601 | TTTTGGATGA ATAAGAGCAC AAGTAAATCT GTACAGATGA TGACACAGAG | |
| 651 | CCATTCCTTT AGCTTCACTT TCCTGGAGGA CTTGCAGGCC AAAATTCTAG | |
| 701 | GGATTCCATA TAAAAACAAC GACCTAAGCA TGTTTGTGCT TCTGCCCAAC | |
| 751 | GACATCGATG GCCTGGAGAA GATAATAGAT AAAATAAGTC CTGAGAAATT | |
| 801 | GGTAGAGTGG ACTAGTCCAG GGCATATGGA AGAAAGAAAG GTGAATCTGC | |
| 851 | ACTTGCCCCG GTTTGAGGTG GAGGACAGTT ACGATCTAGA GGCGGTCCTG | |
| 901 | GCTGCCATGG GGATGGGCGA TCCCTTCAGT GAGCACAAAG CCGACTACTC | |
| 951 | GGGAATGTCG TCAGGCTCCG GGTTGTACGC CCAGAAGTTC CTGCACAGTT | |
| 1001 | CCTTTGTGGC AGTAACTGAG GAAGGCACCG AGGCTGCAGC TGCCACTGGC | |
| 1051 | ATAGGCTTTA CTGTCACATC CGCCCCACGT CATGAAAATG TTCACTGCAA | |
| 1101 | TCATCCCTTC CTGTTCTTCA TCAGGCACAA TGAATCCAAC AGCATCCTCT | |
| 1151 | TCTTCGGCAG ATTTTCTTCT CCTTAA | |

The present invention is based on an unexpected discovery that two SNPs (i.e., 877A/G and 1047T/C) in SERPINB13 Exon 8 are associated with cancer. As demonstrated in the example below, the percentage of heterozygous genotype at both loci is higher among brain cancer patients. In particular, compared with subjects with the 877A/A genotype (i.e., “wild-type”), subjects with the 877A/G genotype (i.e., “variant”) are approximately 2 times more at risk of developing brain cancer. For subjects who are at least 28 years old (the median of age), those with the 877A/G genotype are approximately 3 times more at risk of developing brain cancer than those with the 877A/A genotype. More significantly, for male subjects who are at least 28 years old (the median of age), those with the 877A/G genotype are approximately 4 times more at risk of developing brain cancer than those with the 877A/A genotype. The invention is also based on another unexpected discovery that over-expression of Hurpin protein in invasive or metastatic brain or ovarian tumor cells inhibits invasion or metastasis of the tumor cells.

Accordingly, this invention provides methods for diagnosing and treating cancer associated with SERPINB13 or its SNPs.

Examples of cancer include, but are not limited to, breast cancer, gastrointestinal cancer (e.g., colorectal cancer, esophageal cancer, liver cancer, gallbladder cancer, biliary tract cancer, and pancreatic cancer), genitourinary cancer (e.g., bladder cancer, kidney cancer such as papillary renal cell carcinoma and Von Hippel-Lindau disease, prostate cancer, and testicular cancer), gynecologic cancer (e.g., cervical cancer, endometrial cancer, and ovarian cancer), head and neck cancer (e.g., thyroid cancer), hematologic cancer (leukemia, lymphoma, myelodysplastic syndromes, and multiple myeloma), lung cancer, skin cancer (e.g., melanoma), eye tumor such as retinoblastoma, AIDS-related tumors, brain tumors, endocrine tumors such as multiple endocrine neoplasias, and sarcoma.

In one example, the cancer can have an odds ratio (OR) of 2 with a 95% confidence interval (CI) of 1-12. The odds for the presence of a SERPINB13 SNP are calculated as the number of the occurrences of the SERPINB13 SNP divided by the number of non-occurrences of the SERPINB13 SNP. An OR is calculated by dividing the odds in a cancer group by the odds in a control group.

A diagnostic method of this invention involves preparing a nucleic acid sample (e.g., a tissue sample, a buccal cell sample, or a blood sample) from a subject and identifying an SNP in the SERPINB13 gene. The presence of a single nucleotide polymorphism indicates that the subject is suffering from or at risk for developing cancer. The method of this invention can be used on its own or in conjunction with other procedures to diagnose cancer in appropriate subjects.

A single nucleotide polymorphism (SNP) occurs at a polymorphic site occupied by a single nucleotide, which is the site of variation between allelic sequences. “Polymorphic” refers to the occurrence of two or more genetically determined alternative sequences or alleles in a population of subjects. An SNP usually arises due to substitution, e.g., a transition or transversion, of one nucleotide for another at the polymorphic site. A transition is the replacement of one purine by another purine or one pyrimidine by another pyrimidine. A transversion is the replacement of a purine by a pyrimidine or vice versa. SNPs can also arise from a deletion of a nucleotide or an insertion of a nucleotide relative to a reference allele.

Allele Identification

Methods for allele identification are well known in the art. See, e.g., Ann-Christine Syvanen (2001) Nature Review Genetics 2:930-942. Examples of these methods are described below. They can be used to determine which allele or alleles of the SERPINB13 gene a subject carries. Polymorphisms can be detected in a target nucleic acid from an individual. Samples that contain genomic DNA, cDNA, mRNA, or proteins can be used to determine which of a plurality of polymorphisms are present in a subject.

Amplification of DNA from target samples can be accomplished by methods known to those of skill in the art, e.g., polymerase chain reaction (PCR). See, e.g., U.S. Pat. No. 4,683,202, ligase chain reaction (LCR) (see Wu and Wallace (1989) Genomics 4:560 and Landegren, et al. (1988) Science 241:1077), transcription amplification (Kwoh, et al. (1989) Proc. Natl. Acad. Sci. USA 86:1173), self-sustained sequence replication (Guatelli, et al. (1990) Proc. Nat. Acad. Sci. USA 87:1874), and nucleic acid-based sequence amplification (NASBA).

The methods with which a polymorphism is detected can depend on whether it is known that the polymorphism exists. If it is unknown whether a polymorphism exists, de novo characterization can be employed. This analysis compares target sequences in different individuals to identify points of variation, i.e., polymorphic sites. Analyzing groups of individuals that exhibit high degrees of diversity, e.g., ethnic diversity, allows the identification of patterns characteristic of the most common alleles of the locus. Further, the frequencies of such populations within the population can be determined. Allelic frequencies can be determined for subpopulations characterized by other criteria, e.g., gender.

When it is known that a polymorphism exists, there are a variety of suitable procedures that can be employed to detect the polymorphism, described in further detail below.

Allele-Specific Probes

The design and use of allele-specific probes for analyzing polymorphisms is known in the art (see, e.g., Dattagupta, EP 235,726; and Saiki, WO 89/11548). Allele-specific probes can be designed to hybridize differentially, e.g., to hybridize to a segment of DNA from one individual but not to a corresponding segment from another individual, based on the presence of polymorphic forms of the segment. Relatively stringent hybridization conditions can be utilized to cause a significant difference in hybridization intensity between alleles, and possibly to obtain a condition wherein a probe hybridizes to only one of the alleles. Probes can be designed to hybridize to a segment of DNA such that the polymorphic site aligns with a central position of the probe.

Allele-specific probes can be used in pairs, wherein one member of the pair matches perfectly to a reference form of a target sequence, and the other member of the pair matches perfectly to a variant of the target sequence. The use of several pairs of probes immobilized on the same support may allow simultaneous analysis of multiple polymorphisms within the same target sequence.

Tiling Arrays

Polymorphisms can also be identified by hybridization to nucleic acid arrays (see, e.g., WO 95/11995). WO 95/11995 also describes subarrays that are optimized for detection of a variant form of a precharacterized polymorphism. Such a subarray contains probes designed to be complementary to a second reference sequence, which is an allelic variant of the first reference sequence. The second group of probes is designed to exhibit complementarily to the second reference sequence. The inclusion of a second group (or further groups) can be particular useful for analyzing short subsequences of the primary reference sequence in which multiple mutations are expected to occur within a short distance commensurate with the length of the probes (i.e., two or more mutations within 9 to 21 bases).

Allele-Specific Primers

An allele-specific primer hybridizes to a site on target DNA overlapping a polymorphism and only primers amplification of an allelic form to which the primer exhibits perfect complementarity. See, e.g., Gibbs (1989) Nucleic Acid Res. 17:2427-2448. Such a primer can be used in conjunction with a second primer which hybridizes at a distal site. Amplification proceeds from the two primers leading to a detectable product signifying the particular allelic form is present. A control is usually performed with a second pair of primers, one of which shows a single base mismatch at the polymorphic site and the other of which exhibits perfect complementarity to a distal site. The single-base mismatch prevents amplification and no detectable product is formed. The method can be optimized by including the mismatch at the 3′-most position of the oligonucleotide aligned with the polymorphism because this position is most destabilizing to elongation from the primer. See, e.g., WO 93/22456.

Direct Sequencing

The direct analysis of the sequence of polymorphisms of the present invention can be accomplished using either the dideoxy chain termination method or the Maxam Gilbert method (see Sambrook, et al. (1989) Molecular Cloning, A Laboratory Manual, 2nd Ed., CSHP, New York and Zyskind, et al. (1988) Recombinant DNA Laboratory Manual, Acad. Press).

Denaturing Gradient Gel Glectrophoresis

Amplification products generated in a polymerase chain reaction can be analyzed by the use of denaturing gradient gel electrophoresis. Different alleles can be identified based on the different sequence-dependent melting properties and electrophoretic migration of DNA in solution. See Erlich, ed. (1992) PCR Technology, Chapter 7: Principles and Applications for DNA Amplification, W. H. Freeman and Co, New York.

Single-Strand Conformation Polymorphism Analysis

Alleles of target sequences can be differentiated using single-strand conformation polymorphism analysis, which identifies base differences by alteration in electrophoretic migration of single stranded PCR products, as described in Orita, et al. (1989) Proc. Nat. Acad. Sci. 86:2766-2770. Amplified PCR products can be generated as described above, and heated or otherwise denatured, to form single stranded amplification products. Single-stranded nucleic acids may refold or form secondary structures which are partially dependent on the base sequence. The different electrophoretic mobilities of single-stranded amplification products can be related to base-sequence difference between alleles of target sequences.

This invention also provides a method for treating cancer (e.g., invasive or metastatic cancer, or brain or ovarian cancer) associated with SERPINB13 or its SNPs. Patients to be treated can be identified, for example, by determining the cancer type using methods well known in the art, or by determining the presence of SERPINB13. SNPs in a nucleic acid sample prepared from a patient by the methods described above. If the cancer is invasive or metastatic, or if the SERPINB13 SNPs are present in the nucleic acid sample from the patient, the patient is a candidate for treatment with an effective amount of a compound (e.g., a nucleic acid encoding the Hurpin protein, or the Hurpin protein itself) that increases the Hurpin protein level in the patient.

The treatment method can be performed in vivo or ex vivo, alone or in conjunction with other drugs or therapy.

In one in vivo approach, a therapeutic compound (e.g., a compound that increases the SERPINB13 gene expression level in a cell or the Hurpin protein itself) is administered to the subject. Generally, the compound will be suspended in a pharmaceutically-acceptable carrier (e.g., physiological saline) and administered orally or by intravenous infusion, or injected or implanted subcutaneously, intramuscularly, intrathecally, intraperitoneally, intrarectally, intravaginally, intranasally, intragastrically, intratracheally, or intrapulmonarily.

The dosage required depends on the choice of the route of administration; the nature of the formulation; the nature of the patient's illness; the subject's size, weight, surface area, age, and sex; other drugs being administered; and the judgment of the attending physician. Suitable dosages are in the range of 0.01-100.0 μg/kg. Wide variations in the needed dosage are to be expected in view of the variety of compounds available and the different efficiencies of various routes of administration. For example, oral administration would be expected to require higher dosages than administration by i.v. injection. Variations in these dosage levels can be adjusted using standard empirical routines for optimization as is well understood in the art. Encapsulation of the compound in a suitable delivery vehicle (e.g., polymeric microparticles or implantable devices) may increase the efficiency of delivery, particularly for oral delivery.

Alternatively, a polynucleotide containing a nucleic acid sequence encoding the Hurpin protein can be delivered to the subject, for example, by the use of polymeric, biodegradable microparticle or microcapsule delivery devices known in the art.

Another way to achieve uptake of the nucleic acid is using liposomes, prepared by standard methods. The vectors can be incorporated alone into these delivery vehicles or co-incorporated with tissue-specific antibodies. Alternatively, one can prepare a molecular conjugate composed of a plasmid or other vector attached to poly-L-lysine by electrostatic or covalent forces. Poly-L-lysine binds to a ligand that can bind to a receptor on target cells (Cristiano, et al. (1995) J. Mol. Med. 73:479). Alternatively, tissue specific targeting can be achieved by the use of tissue-specific transcriptional regulatory elements (TRE) which are known in the art. Delivery of “naked DNA” (i.e., without a delivery vehicle) to an intramuscular, intradermal, or subcutaneous site is another means to achieve in vivo expression.

In the relevant polynucleotides (e.g., expression vectors), the nucleic acid sequence encoding the Hurpin protein is operatively linked to a promoter or enhancer-promoter combination. Enhancers provide expression specificity in terms of time, location, and level. Unlike a promoter, an enhancer can function when located at variable distances from the transcription initiation site, provided a promoter is present. An enhancer can also be located downstream of the transcription initiation site.

Suitable expression vectors include plasmids and viral vectors such as herpes viruses, retroviruses, vaccinia viruses, attenuated vaccinia viruses, canary pox viruses, adenoviruses and adeno-associated viruses, among others.

Polynucleotides can be administered in a pharmaceutically acceptable carrier. Pharmaceutically acceptable carriers are biologically compatible vehicles that are suitable for administration to a human, e.g., physiological saline or liposomes. A therapeutically effective amount is an amount of the polynucleotide that is capable of producing a medically desirable result (e.g., an increased level of the Hurpin protein) in a treated patient. As is well known in the medical arts, the dosage for any one patient depends upon many factors, including the patient's size, body surface area, age, the particular compound to be administered, sex, time and route of administration, general health, and other drugs being administered concurrently. Dosages will vary, but a preferred dosage for administration of polynucleotide is from approximately 10⁶ to 10¹² copies of the polynucleotide molecule. This dose can be repeatedly administered, as needed. Routes of administration can be any of those listed above.

An ex vivo strategy for treating cancer patients can involve transfecting or transducing cells obtained from the subject with a polynucleotide encoding the Hurpin protein. Alternatively, a cell can be transfected in vitro with a vector designed to insert, by homologous recombination, a new, active promoter upstream of the transcription start site of the naturally occurring endogenous SERPINB13 gene in the cell's genome. Such methods, which “switch on” an otherwise largely silent gene, are well known in the art. After selection and expansion of a cell that expresses the Hurpin protein at a desired level, the transfected or transduced cells are then returned to the subject. The cells can be any of a wide range of types including, without limitation, neural cells, hemopoietic cells (e.g., bone marrow cells, macrophages, monocytes, dendritic cells, T cells, or B cells), fibroblasts, epithelial cells, endothelial cells, keratinocytes, or muscle cells. Such cells act as a source of the Hurpin protein for as long as they survive in the subject.

The ex vivo methods include the steps of harvesting cells from a subject, culturing the cells, transducing them with an expression vector, and maintaining the cells under conditions suitable for expression of the SERPINB13 gene. These methods are known in the art of molecular biology. The transduction step is accomplished by any standard means used for ex vivo gene therapy, including calcium phosphate, lipofection, electroporation, viral infection, and biolistic gene transfer. Alternatively, liposomes or polymeric microparticles can be used. Cells that have been successfully transduced can then be selected, for example, for expression of the SERPINB13 gene. The cells may then be injected or implanted into the patient.

The specific example below is to be construed as merely illustrative, and not limitative of the remainder of the disclosure in any way whatsoever. Without further elaboration, it is believed that one skilled in the art can, based on the description herein, utilize the present invention to its fullest extent. All publications recited herein are hereby incorporated by reference in their entirety.

Materials and Methods

| | |
|---|---|
| Main Equipments: | LightCycler Real-Time PCR Instrument |
| | (Roche Diagnostics) |
| | Automatic DNA Sequencer (Applied Biosystems) |
| | T3 thermocycler (Whatman Biometra) |
| Primers and Probes: | PCR amplification primers (TIB MOLBIOL or |
| | Genset Oligos) |
| | Hybridization probes (TIB MOLBIOL) |
| | LightCycler DNA master hybridization probes kit |
| | (Roche Diagnostics) |
| Main Reagents: | QIAamp Blood Mini kit (Qiagen Inc.) |
| | BuccalAmp DNA extraction kit (Epicentre |
| | Technology) |
| | LightCycler Color Compensation Set (Roche |
| | Diagnostics) |

Sample Preparation:

A total of 317 subjects were enrolled in this study, including 80 brain cancer patients and 237 healthy controls. Genomic DNA was extracted either from peripheral blood samples using a QIAamp Blood Mini kit or from buccal cells using a BuccalAmp DNA extraction kit according to the manufacturers' protocols. Depending on the quality of the purified genomic DNA, most samples at a range of 10 pg˜500 ng can be directly used as templates for PCR amplification followed by melting curve analysis with the LightCycler Real-Time PCR Instrument. For a few samples of poor quality, an alternative nested-PCR protocol was developed for SNP genotyping. Genotyping of SNPs in SERPINB13 Exon 8:

1. PCR Amplification

Before PCR amplification, in each capillary tube, a volume of 18 μ1 mastermix solution was prepared as shown in Table 1: 10 pmole PrimerS as the sense primer, 0.5 pmole PrimerA as the antisense primer, 0.1 μM Fluorescein SensorG probe, 0.1 M LCred 640 probe, 0.1 μM Fluorescein SensorT probe, 0.1 μM LCred 705 probe, 3 mM MgCl₂, and 1× LightCycler DNA hybridization probe reagent which contains Taq polymerase. The amplification primers and hybridization probes were synthesized by TIB MOLBIOL. After preparation of the mastermix solution, 2 μl of genomic DNA extracted from each individual subject was added to the solution for real-time PCR amplification in a LightCycler rotor. To ensure the quality of PCR, negative and positive controls were included in each set of experiments. Samples for the negative control were prepared by replacing the DNA template with an equal volume of PCR-grade water, whereas samples for the positive control were prepared by replacing the DNA template with a confirmed SERPINB13 cDNA fragment containing two SNP sites (i.e., 877A/G and 1047T/C). PCR was carried out according to the protocol described in Table 2. A 333-bp DNA fragment encompassing the two SNP sites (i.e., 877A/G and 1047T/C) in SERPINB13 Exon 8 was amplified and analyzed using the LightCycler Real-Time PCR Instrument.

TABLE 1
AMPLIFICATION PRIMERS, HYBRIDIZATION PROBES, AND REGENTS

| | Nucleotide | Length | GC | Tm |
|---|---|---|---|---|
| SERPINB13 (DNA sequence in 5′ to 3′ direction) | Position | (mer) | (%) | (° C.) |
| A) Mastermix (total volume = 18 μl) | | | | |
| I. Amplification primers: | | | | |
| 10 pmole PrimerS (GTGGACTAGTCCAGGGCATAT) | 807 | 21 | 52.4 | 54.4 |
| 0.5 pmole PrimerT (TTGGATTCATTGTGCCTGATG) | 1139 | 21 | 42.9 | 56.4 |
| II. Hybridization probes (final concentration: 0.1 μM each): | | | | |
| 1) Fluorescein SensorG probe | 889 | 23 | 56.5 | 59.7 |
| (CTAGATCGTAACCGTCCTCCACCx*) | | | | |
| 2) LCred 640 probe | 865 | 23 | 56.5 | 67.4 |
| (LCred640CAAACCGGGGCAAGTGCAGATTCp*) | | | | |
| 3) Fluorescein SensorT probe | 1063 | 22 | 59.1 | 63.9 |
| (CAGTAAAGCCTATGCCGGTGGCx*) | | | | |
| 4) LCred 705 probe | 1039 | 23 | 65.2 | 69.6 |
| (LCred705CTGCAGCCTCGGTGCCTTCCTCAp*) | | | | |
| III. Reagents: | | | | |
| 1x LightCycler-DNA hybridization probes/polymerase | | | | |
| 3 mM MgCl₂ | | | | |
| PCR grade water (added to a final volume of 18 μl) | | | | |
| B) Genomic DNA (10 pg˜500 ng in 2 μl) | | | | |

x*fluorescein,
p*phosphate labeled

TABLE 2
PCR PROTOCOL
1^{st} step—1 cycle of denaturation at 95° C. for 30 sec
2^{nd} step—45-50 cycles of real-time PCR as shown below:

| | Value | | |
|---|---|---|---|
| | Cycle number | | |
| | 45-50 | | |
| Parameter | Segment 1 | Segment 2 | Segment 3 |
| Temperature target (° C.) | 95 | 60 | 72 |
| Incubation time (sec) | 0 | 10 | 18 |
| Temperature transition rate (° C./sec) | 20 | 20 | 20 |
| Acquisition mode | None | Single | None |

Display mode: F2 (LC640)/F3 (LC705)

2. SNP Genotyping

For LightCycler fluorescent analysis, the Tm values of hybridization probes were purposely designed to be 5-10° C. higher than those of the amplification primers (Table 1). Two sets of hybridization probes were arbitrary designed according to the antisense strand, and complementary to each SNP site at higher Tm values. The first SNP site in SERPINB13 Exon 8 (i.e., 877 A/G) was detected using a 3′-fluorescein (x*)-labeled probe, which was designed to hybridize to a sequence containing the SNP locus (underlined) (5′-CTAGATCGTAACCGTCCTCCACCx*-3′), together with a 5′-LCred640-labeled probe, which was 3′-phosphorylated (p*) to prevent extension during PCR (5′-LCred640CAAACCGGGGCAAGTGCAGATTCp*-3′). The second SNP site (i.e., 1047T/C; underlined) was detected using another fluorescein (x*)-labeled probe (5′-CAGTAAAGCCTATGCCGGTGGCx*-3), together with a 5′-LCred705-labeled probe (5′-LCred705CTGCAGCCTCGGTGCCTTCCTCAp*-3′). It is important to design the hybridization probes such that the Tm of the probe hybridized to the “variant” allele is higher than that of the probe hybridized to the “wild-type” allele. As shown in Table 3, the melting curve analysis for LightCycler fluorescent detection was performed at 95° C. for 0 sec, 45° C. for 60 sec, then gradual increase to 80° C. (0.1° C./sec) followed by final cooling of 30 sec at 40° C. To ensure the quality of SNP genotyping, in each set of experiments, at least one PCR-amplified sample was resolved by agarose gel electrophoresis followed by gel purification and automated DNA sequencing to double check the efficacy and accuracy of the LightCycler fluorescent detection method.

TABLE 3
MELTING CURVE ANALYSIS

| | Value | | |
|---|---|---|---|
| | Cycle number | | |
| | 1 | | |
| | Type | | |
| | Melting curve | | |
| Parameter | Segment 1 | Segment 2 | Segment 3 |
| Temperature target (° C.) | 95 | 45 | 80 |
| Incubation time (sec) | 0 | 60 | 0 |
| Temperature transition rate | 20 | 20 | 0.1 |
| (° C./sec) | | | |
| Acquisition mode | None | None | Continuous |

Display mode: F2 (LC640)/F3 (LC705)
(Final cooling: 30 sec at 40° C.)

3. Alternative PCR Protocol for Samples of Poor Quality

During the SNP genotyping analysis, some genomic DNA samples failed to be amplified by real-time PCR. Thus, an alternative nested PCR protocol was developed to amplify these genomic DNA samples. Two gene-specific primers, designated Exon 8-Downout (5′-TGAGAAATTGGTAGAGTGGACTAGT-3′) as the sense primer and Tail-Upout (5′-CAGCAATGCCATGGCAACGATCAT-3′) as the antisense primer, were used for the first round of PCR. PCR was performed in a T3 thermocycler using Taq polymerase with 1 cycle of 94° C. for 2 min; 35 cycles of 94° C. for 30 sec, 55° C. for 30 sec, and 72° C. for 1 min; and 1 cycle of 72° C. for 10 min. After the reaction, 1 μl of a 1:25 dilution of the primary PCR products was subjected to a second round of PCR, with a pair of gene-specific primers named Exon 8-Downest (5′-CTAGTCCAGGGCATATGGAAGAA-3′) as the sense primer and Tail-Upnest (5′-GAGAAGAAAATCTGCCGAAGAAGA-3′) as the antisense primer, to amplify a final 360-bp DNA fragment encompassing the two SERPINB13 SNP sites (i.e., 877A/G and 1047T/C). Nested PCR was carried out under the following conditions: 1 cycle of 94° C. for 2 min; 45 cycles of 94° C. for 30 sec, 55° C. for 30 sec, and 72° C. for 1 min; and 1 final cycle of 72° C. for 10 min. All of the Taq-amplified products were resolved by agarose gel electrophoresis. Each amplified DNA fragment was then gel-purified either for automated DNA sequencing using Exon 8-Upnest primer, or by LightCycler fluorescent analysis as described above.

Detection of SERPINB13 Exon 8 SNPs in cDNA Libraries:

DNA templates used for the detection of sequence variations in the SERPINB13 gene were from 4 different cDNA libraries: the brain MATCHMAKER cDNA library, pancreas and testis 5 ′-STRETCH PLUS cDNA libraries (Clontech Inc.), and duodenum GENE POOL cDNA library (Invitrogen). PCR was performed in the T3 thermocycler using the sense primer (5′-ATGGATTCACTTGGCGCCGTCAGCAC-3′), the antisense primer (5′-TTAAGGAGAAGAAAATCTGCCGAAG-3′), and high-fidelity Vent polymerase (New England Biolabs) under the following conditions: 1 cycle of 94° C. for 3 min; 40 cycles of 94° C. for 30 sec, 55° C. for 30 sec, and 72° C. for 2 min; and 1 cycle of 72° C. for 10 min. The amplified fragment containing the open reading frame of SERPINB13 was then resolved by 0.8% (w/v) agarose gel electrophoresis, purified using a QIAquick gel purification kit (Qiagen Inc.), and cloned into pCRII vectors using the one-step TOPO TA cloning strategy (Invitrogen). After blue/white colony screening, cells were cultured overnight at 37° C. in LB broth containing 50 μg/ml ampicillin. Plasmid DNA was isolated from bacterial cultures using the miniprep extraction kit (Qiagen Inc.), and inserts were confirmed by EcoR I enzyme digestion (New England Biolabs) followed by agarose gel electrophoresis. The Sp6 and T7 primers were applied to the purified plasmids for automated DNA sequencing.

Cell Culture:

Three high-invasive human cancer cell lines, brain glioblastoma U-87MG cells, brain neuroglioma H4 cells, and ovarian adenocarcinoma A59-4 cells were used in this study. The U-87MG cells were cultured in MEM medium (Gibco BRL) containing Earle's salts, 2 mM L-glutamine, 0.1 mM non-essential amino acid, and 1 mM sodium pyruvate supplemented with 10% heat-inactivated fetal bovine serum (FBS). The H4 and A59-4 cells were cultured in DMEM medium (Gibco BRL) containing 4.5 g/L glucose supplemented with 10% heat-inactivated FBS. All three cell lines were cultured at 37° C. humidified incubator with 5% CO₂.

Cloning and Site-Directed Mutagenesis of SERPINB13:

Two primers, designated SerpB13-EGFP-head primer (5′-CTTCGAATTCGTATGGATTCACTTGG-3′with an EcoR I restriction site (GAATTC) added at the 5 ′-end) as the sense primer and SerpB 13-EGFP-tail prmier (5′-GTGGATCCTGAGGAGAAGAAAA-3′with a BamH I restriction site (GGATCC) added at the 5′-end) as the antisense primer, were used for PCR amplification and cloning. First, PCR was performed using ExTaq polymerase (Takara Shuzo Co.) with 1 cycle at 94° C. for 2 min; 35 cycles at 94° C. for 30 sec, 55° C. for 30 sec, and 72° C. for 1 min; and 1 cycle at 72° C. for 10 min. After amplification, the amplicon and mammalian pEGFP-N1 expression vector (Clontech Inc.) were double digested with EcoR I and BamH I restriction enzymes followed by ligation with T4 DNA ligase (Promega Corp.) and E. coli transformation. Plasmid DNA was isolated from bacterial cultures using the miniprep extraction kit, and inserts were confirmed by EcoR I and BamH I enzyme digestion as well as automated DNA sequencing.

To change the 293th amino acid residue of the Hurpin protein from serine (i.e., “wild-type”) to glycine (i.e., “variant”), two primers, the forward primer (SerB13-Gly-Fwd, 5′-GAGGTGGAGGACGGTTACGATCTAGAGGC-3′) and the reverse primer (SerB13-Gly-Rev,5′-GCCTCTAGATCGTAACCGTCCTCCACCTC-3′), were designed and a QuickChange site-directed mutagenesis kit (Stratagene) was utilized according to the manufacturer's protocol. Using the wild-type SERPINB13 in pEGFP-N1 as a template, PCR was performed with 1 cycle at 95° C. for 30 sec; 12 cycles at 95° C. for 30 sec, 55° C. for 1 min; and 1 cycle at 68° C. for 12 min followed by Dpn I treatment to digest parental wild-type DNA template. Finally, the nicked vector DNA containing the desired mutation was transformed and grown in XL1-Blue cells, isolated using miniprep extraction kit, and verified by DNA autosequencing for the desired amino acid change.

Transfection and Over-Expression of Wild-Type and Variant SERPINB13 in Metastatic Cancer Cells:

Before the day of transfection, according to the size and growth of each metastatic cancer cell line, 2-2.8×10⁶ cells were seeded in a 10 cm culture dish so that they were 90% confluent on the day of transfection. DNA was transfected using the Lipofectamine Plus Reagent (Invitrogen) according to the manufacturer's protocols. The amount of DNA used for gene transfection was 4 μg of pEGFP-N1 vector constructed with or without wild-type or variant SERPINB13.

Immunoblot Analysis of Hurpin Protein Over-Expression in Metastatic Cancer Cells:

After transfection, cells transfected with SERPINB13 were selected with G418 for several weeks. The insertion and expression of the SERPINB313/EGFP fusion protein were then visualized under a fluorescence microscope. To analyze the Hurpin protein expression level, the metastatic cancer cells were harvested and lysed at 4° C. for 15 min in 350 μl radioimmunoprecipitation buffer containing 1 μg/ml aprotinin, 1 mM leupeptin, 1 mM Na₃VO₄, 1 mM NaF and 1 mM PMSF. After centrifugation at 16,000×g for 30 min at 4° C., the cell lysates (supernatant) were collected and an equal amount of proteins (50 μg) from each sample was separated by 12% (w/v) sodium dodecyl sulfate-polyacrylamide gel electrophoresis (SDS-PAGE). The gel was equilibrated in a transfer buffer at room temperature, and the proteins were transferred onto polyvinylidene fluoride membranes (Millipore) for 2 hr at 4° C. The membranes were then blocked with 5% (w/v) non-fat dry milk in TBST (12.5 mM Tris/HCl, pH 7.6, 137 mM NaCl, 0.05% Tween 20) at room temperature for 1 h. The membranes were then washed with TBST, and blots were individually incubated with mouse anti-EGFP (Clontech Inc.) or anti-tubulin (NeoMarkers Inc.) monoclonal antibody for 1 hr at room temperature. The membranes were washed 2 times with TBST, 15 min each, followed by incubation with HRP-labeled secondary antibodies for 1 hr at room temperature. After another 2 washes, bands were visualized with the SuperSignal West Pico Stable Peroxide Solution (Pierce) and exposed to X-ray film (Midwest Scientific).

Invasion Assay:

To evaluate the invasiveness of tumor cells, the membrane invasion culture system (MICS) was applied as described previously (Hendrix, et al. (1987) Cancer Lett. 38:137). Briefly, a polycarbonate membrane containing 10 μm pores (Nucleopore Corp.) was coated with a reconstituted basement-membrane matrigel (BD Biosciences). The membrane was placed between the upper- and lower-well plates of the MICS chamber. Various metastatic cancer cells were then resuspended and seeded into the upper wells of the chamber (2.5×10⁴ cells/well). After incubation for 48 hr at 37° C., cells that had invaded were harvested from the lower wells with 1 mM ethylene diamine tetraacetic acid in phosphate buffered saline, and dot-blotted on a 3 μm polycarbonate membrane. After fixation in pure methanol, blotted cells were stained with 50 μg/ml propidium iodine (PI) for 30 min and the cell number in each blot was counted under a light microscope at a magnification of 25× using the Analytical Imaging Station software package (Imaging Research Inc.). Each experiment was performed 3 times and each sample was assayed in octaplicate.

Results

A population-based case-control study was performed to identify the association of two SERPINB13 single nucleotide polymorphisms (i.e., 877A/G and 1047T/C) with cancer. By genotyping a total of 317 subjects including 80 brain cancer patients and 237 healthy controls, it was found, unexpectedly, that 18.57% of the control subjects and 28.75% of the brain cancer patients were heterozygous at both loci (i.e., AG/TC) (Table 4). This result indicates that the two SERPINB13 Exon 8 SNPs are high-frequency SNPs. Compared to subjects of the 877A/A (i.e., “wild-type”) genotype, as shown in Table 5, subjects of the 877A/G (i.e., “variant”) genotype had an elevated risk of brain cancer (OR=2.00, 95% CI=1.09−3.68, P=0.0263). In particular, for subjects who were at least 28 years old (the median of age), those with the 877A/G genotype were approximately 3 times more at risk of developing brain cancer than those with the 877A/A genotype (OR=2.59, 95% CI=1.20−5.58, P=0.0002). More significantly, for male subjects who were at least 28 years old (the median of age), those with the 877A/G genotype were approximately 4 times more at risk of developing brain cancer than those with the 877A/A genotype (OR=3.77, 95% CI=1.18−12.00, P=0.0060).

TABLE 4
FREQUENCIES OF SERPINB13 EXON 8 877/1047 ALLELES

| | Genotype: 1^{st} allele (877A/G) and 2^{nd} allele (1047T/C) | | |
|---|---|---|---|
| | | | Homozygous |
| | Homozygous alleles | Heterozygous alleles | alleles^{¶} |
| | (877AA/1047TT) | (877AG/1047TC) | (877GG/1047CC) |
| Total | | | |
| (n = 317) | | | |
| Controls | 192 (81.01%) | 44 (18.57%) | 1 (0.42%) |
| (n = 237) | | | |
| Male | 92 | 22 | 0 |
| Female | 100 | 22 | 1 |
| Cases | 57 (71.25%) | 23 (28.75%) | 0 (0.0%) |
| (n = 80) | | | |
| Male | 26 | 13 | 0 |
| Female | 31 | 10 | 0 |

^{¶}Not included in further analyses.

TABLE 5
RISK OF BRAIN CANCER ASSOCIATED WITH SERPINB13 EXON 8 877
ALLELES

| | Controls | Cases | Odds Ratio | |
|---|---|---|---|---|
| | n (%) | n (%) | (95% CI) | P value |
| Total subjects (n = 316) | | | | |
| Sex (male vs. female) | | | 0.93 (0.55-1.59) | 0.7977 |
| Age (yr) | | | 1.03 (1.02-1.05) | <0.0001 |
| Genotype (variant vs. wild type) | | | 2.00 (1.09-3.68) | 0.0263 |
| Age; genotype | | | | |
| ≧28 yr; wild type (A/A) | 88 (70.97%) | 36 (29.03%) | 1.00 (Referent) | |
| ≧28 yr; variant (A/G) | 17 (48.57%) | 18 (51.43%) | 2.59 (1.20-5.58) | 0.0002 |
| <28 yr; wild type (A/A) | 104 (83.20%) | 21 (16.80%) | 0.49 (0.27-0.91) | 0.0167 |
| <28 yr; variant (A/G) | 27 (84.38%) | 5 (15.63%) | 0.45 (0.16-1.27) | 0.0867 |
| Male (n = 153) | | | | |
| Age; genotype | | | | |
| ≧28 yr; wild type (A/A) | 41 (74.55%) | 14 (25.45%) | 1.00 (Referent) | |
| ≧28 yr; variant (A/G) | 7 (43.75%) | 9 (56.25%) | 3.77 (1.18-12.00) | 0.0060 |
| <28 yr; wild type (A/A) | 51 (80.95%) | 12 (19.05%) | 0.69 (0.29-1.65) | 0.0819 |
| <28 yr; variant (A/G) | 15 (78.95%) | 4 (21.05%) | 0.78 (0.22-2.75) | 0.3509 |
| Female (n = 163) | | | | |
| Age; genotype | | | | |
| ≧28 yr; wild type (A/A) | 47 (68.12%) | 22 (31.88%) | 1.00 (Referent) | |
| ≧28 yr; variant (A/G) | 10 (52.63%) | 9 (47.37%) | 1.92 (0.68-5.40) | 0.0084 |
| <28 yr; wild type (A/A) | 53 (85.48%) | 9 (14.52%) | 0.36 (0.15-0.87) | 0.2156 |
| <28 yr; variant (A/G) | 12 (92.31%) | 1 (7.69%) | 0.18 (0.02-1.46) | 0.1307 |

To examine whether SERPINB13 possesses anti-invasive or anti-metastatic ability, the open reading frame of SERPINB13 cDNA was amplified and cloned into a mammalian expression vector (pEGFP-N1) under the control of the CMV promoter with codon-usage preferences. Unexpectedly, compared to high-invasive tumor cells (U87-MG, H4, and A59-4) transfected with pEGFP-N1 vector alone, cells over-expressing wild-type SERPINB13 significantly decreased in the invasive ability. These results clearly demonstrate the importance of SERPINB13 in tumor invasion and suggest its potential application for metastasis inhibition. In addition, over-expression of a Hurpin protein containing an amino acid change at the 293th amino acid residue from serine (i.e., “wild-type”) to glycine (i.e., “variant”) also decreased the invasive ability of cancer cells, though, in the case of U87-MG metastatic cells, only 25% of decrease was observed.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A method of determining whether a subject is suffering from or at risk for developing cancer, the method comprising:
preparing a nucleic acid sample from a subject, and
identifying a single nucleotide polymorphism in a SERPINB13 gene; wherein the presence of a single nucleotide polymorphism indicates that the subject is suffering from or at risk for developing cancer.

2. The method of claim 1, wherein the single nucleotide polymorphism occurs at a position corresponding to nucleotide 877 of SEQ ID NO:1.

3. The method of claim 2, wherein a guanine, thymine, or cytosine is present at the position corresponding to nucleotide 877 of SEQ ID NO:1.

4. The method of claim 3, wherein a guanine is present at the position corresponding to nucleotide 877 of SEQ ID NO:1.

5. The method of claim 4, wherein the cancer is brain cancer.

6. The method of claim 5, wherein the subject is at least twenty-eight years old.

7. The method of claim 6, wherein the subject is a male.

8. The method of claim 2, wherein another single nucleotide polymorphism occurs at a position corresponding to nucleotide 1047 of SEQ ID NO:1.

9. The method of claim 8, wherein a cytosine, adenine, or guanine is present at the position corresponding to nucleotide 1047 of SEQ ID NO:1.

10. The method of claim 9, wherein a cytosine is present at the position corresponding to nucleotide 1047 of SEQ ID NO:1.

11. The method of claim 10, wherein the cancer is brain cancer.

12. The method of claim 1, wherein the single nucleotide polymorphism occurs at a position corresponding to nucleotide 1047 of SEQ ID NO:1.

13. The method of claim 12, wherein a cytosine, adenine, or guanine is present at the position corresponding to nucleotide 1047 of SEQ ID NO:1.

14. The method of claim 13, wherein a cytosine is present at the position corresponding to nucleotide 1047 of SEQ ID NO:1.

15. The method of claim 14, wherein the cancer is brain cancer.

16. A method of treating brain or ovarian cancer, the method comprising administering to a subject in need thereof an effective amount of a nucleic acid containing SEQ ID NO:1, thereby providing a functional Hurpin protein.

17. The method of claim 16, wherein the cancer is invasive or metastatic.

18. A method of treating brain or ovarian cancer, the method comprising administering to a subject in need thereof an effective amount of a polypeptide encoded by SEQ ID NO:1.

19. The method of claim 18, wherein the cancer is invasive or metastatic.

20. A method of treating invasive or metastatic cancer, the method comprising administering to a subject in need thereof an effective amount of a nucleic acid containing SEQ ID NO:1, thereby providing a functional Hurpin protein.

21. A method of treating invasive or metastatic cancer, the method comprising administering to a subject in need thereof an effective amount of a polypeptide encoded by SEQ ID NO:1.
